(19) United States
(12) Patent Application Publication
(10) Pub. No.: US 2008/0125773 A1
(43) Pub. Date: May 29, 2008

(54) ELECTRODE ASSEMBLY OF ELECTROSURGICAL KNIFE

(76) Inventors: Cheng-Wei Yeh, Hsin Tien City (TW); Yi-Pen Chou, Hsin Tien City (TW)

Correspondence Address: HDSL, 4331 STEVENS BATTLE LANE, FAIRFAX, VA, 22033, US

(21) Appl. No.: 11/847,631

(22) Filed: Aug. 30, 2007

(30) Foreign Application Priority Data
Nov. 24, 2006 (TW) 095220765

(51) Int. Cl. A61B 18/18

(52) U.S. Cl. 606/45

(57) ABSTRACT

An electrode assembly of an electrosurgical knife in which a metallic conduit is used as a conductive passage is provided on an outer sleeve of the electrosurgical knife. The electrode assembly includes a metallic conduit. The rear end of the metallic conduit is connected with a conduit within the outer sleeve. The outer periphery of a front end of the metallic conduit is provided with an insulating sleeve. The insulating sleeve is assembled with the mouth of the outer sleeve. Further, the mouth of the metallic conduit is provided with an electrode block and is connected with the insulating sleeve. Finally, the outer sleeve is provided with an electrode lead therein. The lead is electrically connected with the metallic conduit. With the electrode conduit, an external power supply can be conducted to the electrode block via the metallic conduit, so that the electrode assembly of the electrosurgical knife can perform a normal action. In this way, the convenience in assembling the electrosurgical knife can be improved.

### BACKGROUND OF THE INVENTION

1. Field of the Invention

The present invention relates to an electrosurgical knife, and in particular to an electrosurgical knife capable of vaporizing tissues of a human body.

2. Description of Prior Art

With the progress of technology, medical processes or medical instruments also improve and update in synchronization with the development of technology. Further, the treatments for various kinds of diseases are continuously innovated and improved, one of which is electrosurgical operation. The electrosurgical operation utilizes a current flowing through tissues of a human body to cause tissue impedance, thereby generating high heat to achieve a cutting operation or a hemostatic action. Therefore, the electrosurgical operation is widely used in general surgery, ophthalmic surgery and even plastic surgery, which has become one of the most common operations.

The tool used in an electrosurgical operation is called an electrosurgical knife. A current is introduced to the electrosurgical knife, so that the portion of the electrosurgical knife contacting the tissue of the human body can form an electrode end, thereby performing an operation. Since the efficient introduction of the current is crucial to the electrosurgical knife. In an electrode assembly of a current conventional electrosurgical knife, as shown in FIG. 1, the electrode assembly of the electrosurgical knife 10 includes a conduit 101 for guiding the flow of a saline solution. The outer periphery of the conduit 101 is provided with an insulating sleeve 102. The mouth of the conduit 101 is provided thereon with an electrode block 103 that is connected with the insulating sleeve 102. Finally, an electrode pillar 104 extends from the electrode block 103 to penetrate through the insulating sleeve 102. With an electrode lead 105 for introducing a power supply being soldered onto the electrode pillar 104, the electrode lead 105 is electrically connected with the electrode block 103 via the electrode pillar 104, thereby providing electricity for the electrosurgical knife 10.

However, in the above-mentioned conventional structure, in order to the electrically connect the electrode lead 105 with the electrode block 103, it is necessary to provide an electrode pillar 104 extending from the electrode block 103 to act as an electrode contact while penetrating the insulating sleeve 102. In this way, the electrode lead 105 and the electrode pillar 104 can be soldered together to form an electrical connection. However, such process not only makes the production of the electrosurgical knife 10 too complicated, but also the structure of the electrosurgical knife 10 cannot be made more compact. Further, fixing the electrode lead 105 to the electrode pillar 104 by means of soldering is prone to get broken due to external forces, which affects the efficient delivery of electricity. The above drawbacks have already made the electrode assembly of the electrosurgical knife 10 to be improved necessarily.

### SUMMARY OF THE INVENTION

In view of the above drawbacks, the present invention provides an electrode assembly of an electrosurgical knife in which a metallic conduit is used as a conductive passage. By providing a metallic conduit in the electrode assembly of the electrosurgical knife and connecting with the electrode block, the electrode lead and the metallic conduit can be electrically connected with each other. Further, with the metallic conduit acting as a conductive passage, electricity can be supplied to the electrode block so as to perform a normal action.

The present invention is to provide an electrode assembly of an electrosurgical knife, which is provided on an outer sleeve of the electrosurgical knife. The electrode assembly includes a metallic conduit. The rear end of the metallic conduit is connected with a conduit within the outer sleeve. The outer periphery of the front end of the metallic conduit is provided with an insulating sleeve. The insulating sleeve is assembled with the mouth of the outer sleeve. Further, the mouth of the metallic conduit is provided with an electrode block and is connected with the insulating sleeve. Finally, the outer sleeve is provided with an electrode lead therein. The lead is electrically connected with the metallic conduit. With the electrode conduit, an external power supply can be conducted to the electrode block via the metallic conduit, so that the electrode assembly of the electrosurgical knife can perform a normal action. In this way, the convenience in assembling the electrosurgical knife can be improved.

### BRIEF DESCRIPTION OF THE DRAWINGS

FIG. 1 is a cross-sectional view showing the structure of prior art;

FIG. 2 is a perspective view showing the structure of the present invention;

FIG. 3 is a cross-sectional view showing the structure of the present invention;

FIG. 4 is a cross-sectional view showing the structure of another embodiment of the present invention; and

FIG. 5 is a cross-sectional view showing the structure of a further embodiment of the present invention.

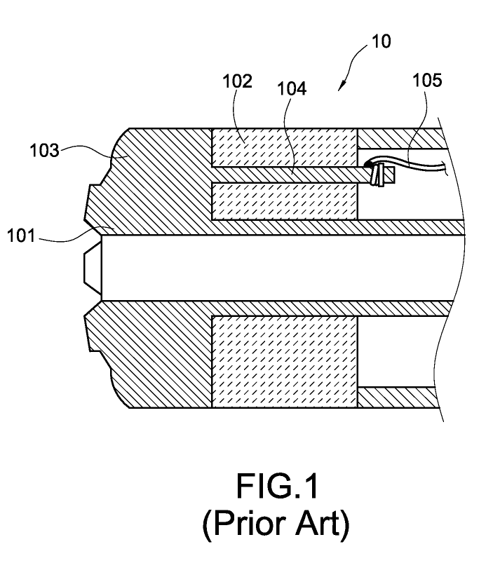

### DETAILED DESCRIPTION OF THE INVENTION

The technical contents of the present invention will be described with reference to the accompanying drawings.

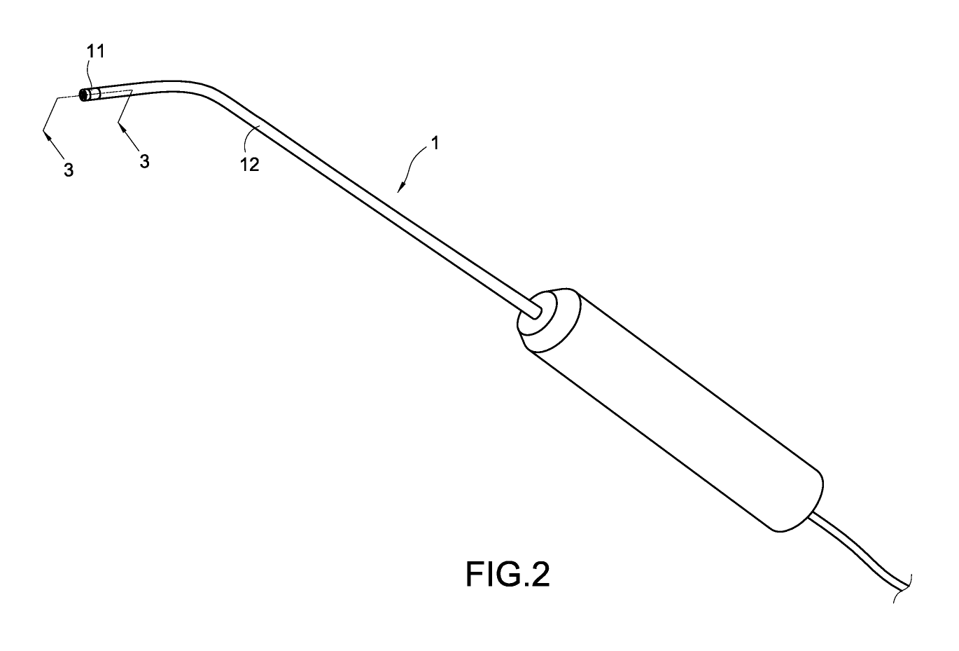

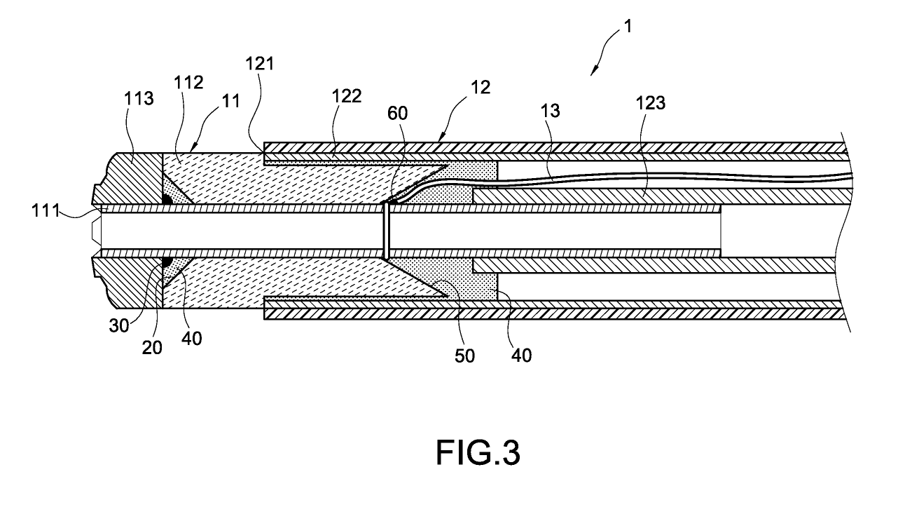

FIG. 2 and FIG. 3 are the perspective view and the cross-sectional view showing the structure of the present invention respectively. As shown in these figures, the electrode assembly 11 of the present invention is provided in a mouth 121 of an outer sleeve 12 of an electrosurgical knife 1. The inner edge of the outer sleeve 12 is provided with a metallic tube 122. The electrode assembly 11 includes a metallic conduit 111 that is made of conductive metallic materials. The rear end of the metallic conduit 111 is connected with a conduit 123 that is positioned within the outer sleeve 12 and accommodated in the metallic tube 122, thereby guiding the saline solution flowing from the outer sleeve 12 to the corresponding tissues of a human body. The conduit 123 is made of an insulating material. Further, the outer periphery of the front end of the metallic conduit 111 is provided with an insulating sleeve 112 that is made of ceramic materials so as to insulate against the electrical conduction between the metallic conduit 111 and the metallic tube 122. The insulating sleeve 112 is connected with the mouth 121 of the outer sleeve 12. Further, the position at which the insulating sleeve 112 abuts against the peripheral wall face of the front end of the metallic conduit 111 is formed with an annular chamfer, thereby forming an annular space 20. The mouth of the metallic conduit 111 is provided thereon with an electrode block 113 that is made of conductive materials. In the present embodiment, the electrode block 113 is provided on the mouth of the metallic conduit 111 and is connected with the insulating sleeve 112. In the annular space 20, the contacting position between the metallic conduit 111 and the electrode block 113 is formed with a soldering point 30 by means of soldering technology, thereby fixing the position of the metallic conduit 111 with respect to the electrode block 113. Finally, an insulating material 40 such as silicone is used to fill the annular space 20. Further, in the present embodiment, the front end of the electrode block 113 is located in a position that is substantially higher than that of the mouth of the metallic conduit 111. Finally, the outer sleeve 12 is provided with an electrode lead 13 therein that is electrically connected on the outer periphery of the metallic conduit 111 and located between the conduit 123 and the insulating sleeve 112. The position at which the insulating sleeve 112 abuts against the peripheral wall face of the rear end of the metallic conduit 111 is formed with an annular chamfer, thereby forming an annular space 50. In the present embodiment, the electrode lead 13 is wound around the innermost end of the annular space 50 to fix the position of the electrode lead 13 with respect to the metallic conduit 111. Via soldering technology (such as laser soldering), the electrode lead 13 is soldered onto the metallic conduit 111 to form a soldering point 60. Finally, the insulating material 40 is used to fill the annular space 50. In this manner, the electrode lead 13 guides the external power supply to the electrode block 113 via the metallic conduit 111, so that the metallic conduit 111 forms a substantially conductive passage, thereby providing electricity for the electrode assembly 11 of the electrosurgical knife 1 to perform a normal action.

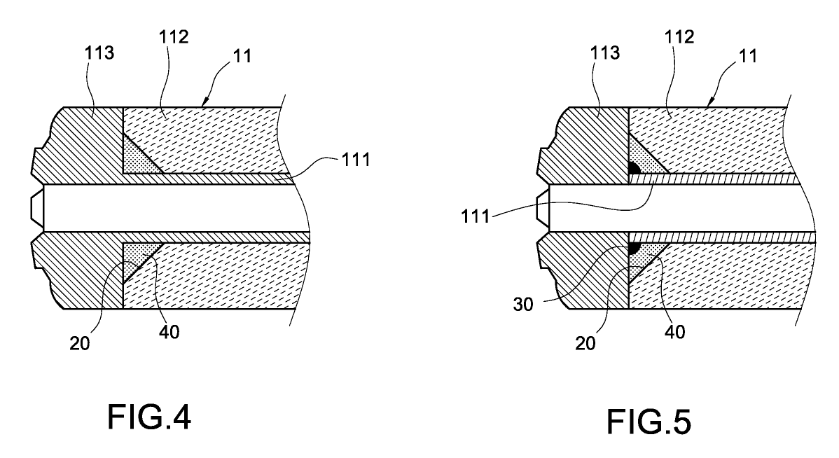

FIG. 4 is a cross-sectional view showing the structure of another embodiment of the present invention, and FIG. 5 is a cross-sectional view showing the structure of a further embodiment of the present invention. As shown in FIG. 4, in addition to the previous manner in which the electrode block 113 is provided on the metallic conduit 111, the metallic conduit 111 can be integrally formed with the electrode block 113. Further, among the electrode block 113, the metallic conduit 111 and the insulating sleeve 112, an annular space 20 is formed. An insulating material 40 is used to fill the annular space, thereby increasing the strength in structure. In this way, the electrode lead 13 electrically connected with the metallic conduit 111 can conduct the electricity to the electrode block 113 directly. Further, as shown in FIG. 5, the end face of the electrode block 113 abuts against the end face of the mouth of the metallic conduit 111. Further, the position at which the insulating sleeve 112 abuts against the peripheral wall face of the front end of the metallic conduit 111 is formed with an annular chamfer, thereby forming an annular space 20. Via the soldering technology, the contacting position between the metallic conduit 111 and the electrode block 113 forms a soldering point 30 to fix the metallic conduit 111 and the electrode block 113, so that the electrode lead 13 electrically connected with the metallic conduit 111 can conduct the power supply to the electrode block 113 via the metallic conduit 111.

Although the present invention has been described with reference to the foregoing preferred embodiments, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims.

What is claimed is:

1. An electrode assembly provided on an outer sleeve of an electrosurgical knife, comprising:
a metallic conduit, one end thereof being assembled with the outer sleeve;
an insulating sleeve provided on an outer periphery of the metallic conduit and connected with the outer sleeve;
an electrode block provided on a mouth of the metallic conduit and assembled with the insulating sleeve; and
an electrode lead assembled with the metallic conduit to form an electrical connection therewith, so that the metallic conduit forms a substantially conductive passage to supply the electricity to the electrode block.

2. The electrode assembly according to claim 1, wherein the electrode lead is connected with the metallic conduit by means of soldering technology to form a soldering point.

3. The electrode assembly according to claim 2, wherein an rear end of the insulating sleeve is formed with an annular chamfer to form an annular space, and the soldering point between the electrode lead and the metallic conduit is provided at an innermost end of the annular space.

4. The electrode assembly according to claim 3, wherein the annular space is filled with an insulating material therein.

5. The electrode assembly according to claim 1, wherein an inner edge of the outer sleeve of the electrosurgical knife is further provided with a metallic tube, the metallic tube is provided therein with a conduit, and the conduit is connected with one end of the metallic conduit.

6. The electrode assembly according to claim 1, wherein the electrode block is provided at the periphery of the mouth of the metallic conduit.

7. The electrode assembly according to claim 6, wherein the front end of the insulating sleeve is formed with an annular chamfer to form an annular space.

8. The electrode assembly according to claim 7, wherein the contacting position between the conductive block and the metallic conduit in the annular space has a soldering point formed by the soldering technology.

9. The electrode assembly according to claim 8, wherein the annular space is filled therein with an insulating material.

10. The electrode assembly according to claim 1, wherein the metallic conduit is integrally formed with the electrode block.

11. The electrode assembly according to claim 10, wherein the front end of the insulating sleeve provided on the metallic conduit is formed with an annular chamfer to form an annular space.

12. The electrode assembly according to claim 11, wherein the annular space is filled therein with an insulating material.

13. The electrode assembly according to claim 1, wherein an end face of the electrode block abuts against an end face of the mouth of the metallic conduit.

14. The electrode assembly according to claim 13, wherein the front end of the insulating sleeve is formed with an annular chamfer to form an annular space.

15. The electrode assembly according to claim 14, wherein the contacting position between the conductive block and the metallic conduit in the annular space has a soldering point formed by the soldering technology.

16. The electrode assembly according to claim 15, wherein the annular space is filled therein with an insulating material.
